(19) United States
(12) Patent Application Publication
(10) Pub. No.: US 2008/0184594 A1
(43) Pub. Date: Aug. 7, 2008

(54) Shoe Sole With Pressure Massage Function

(76) Inventor: Stephan Ebeling, Dusseldorf (DE)

Correspondence Address: Reising, Ethington, Barnes, Kisselle, P.O.Box 4390, Troy, MI, 48099-4390, US

(21) Appl. No.: 11/665,599

(22) PCT Filed: Oct. 26, 2005
(86) PCT No.: PCT/EP05/11464

§ 371 Date: Nov. 5, 2007

(30) Foreign Application Priority Data
Oct. 29, 2004 (DE) 10 2004 052 935.3

(51) Int. Cl. A43B 3/12; A43B 13/18; A61F 5/01

(52) U.S. Cl. 36/11.5; 36/30 R; 36/141

(57) ABSTRACT

The invention relates to a shoe sole (1) that features a pressure massage function and comprises a bottom outsole (20), a top covering sole (40), and at least one adjustable pressure massage element (10) that is located between the outsole (20) and the covering sole (40). Said pressure massage element (10) is accessible from below through the outsole (20) via a breakthrough (21) in the outsole (20) in order to adjust the pressure massage element (10). The pressure massage element (10) is provided with a bolt (11) which extends perpendicular to the shoe sole (1) and is used for applying the pressure massage to the foot. In order to be able to activate and deactivate the massage function in a simple manner, the bolt (11) is vertically adjustable at least between a lower inactive position and an upper active position in which the bolt (11) causes the covering sole (40) to rise and thus the pressure massage to be applied to the foot.

### FIELD OF THE INVENTION

The invention relates generally to a shoe sole with a pressure massage function. In particular, the invention relates to a shoe sole with a pressure massage function with a pressure massage element which is accessible and adjustable from below through an outsole. The invention also relates to a shoe and a sandal.

### BACKGROUND OF THE INVENTION

Generally, shoe soles are made up of more than one layer comprising at least one lower outsole and an insole. In this case, the insole is in contact with the foot. There is often provided also a mid-sole between the insole and the outsole.

The parts are normally firmly connected to one another, each part having specific adapted properties—adapted for its function and for the area of use of the shoe sole with the resultant requirements. The properties are defined by the selection of the materials used and by the shaping of the parts.

By modification of the construction and by incorporation of additional elements, shoe soles can be provided with additional functionality. For instance, measures for foot massage or for additional ventilation of the shoe through to the specific incorporation of active medical substances are known.

Foot massage is of particular significance here. The literature therefore discloses measures which range from massage over a surface area, which enhances the general feeling of well-being, to a specific massage of individual regions of the foot in the sense of foot reflex zone massage.

For instance, DE 101 41 568 discloses a shoe with a massaging action, in which a massage element is incorporated between a flexible base plate and an elastic protective nonwoven firmly bonded to it. The massage element comprises a multiplicity of protuberances or beads, which are arranged such that they are distributed to the greatest extent over the entire tread.

Such systems or systems of a similar construction with a multiplicity of massage protuberances or elements make it possible for the sole of the foot to be massaged over a surface area, with a consequent increase in the blood circulation. Furthermore, the protuberances improve the air circulation between the sole of the foot and the sole of the shoe.

However, a specific massage, designed for the individual needs of the wearer of the shoe, similar to a foot reflex zone massage, is not possible with such a construction. Although the massage element can be differently configured in the various regions of the foot bed, the multiplicity of massage elements and the fact that the massage elements cannot be individually adapted rather leads to a general stimulation in the entire region of the sole of the foot and a rather undifferentiated massage of all the foot reflex zones.

At the same time, foot reflex zone massage is also a method of healing that is still treated with caution by orthodox medicine, but the effectiveness of which has, according to its proponents, been confirmed in practice. For instance, the various regions on the soles of the feet, that is the foot reflex zones, can be respectively assigned organs that can be influenced by specific massage of the respective zone, that is to say the exertion of pressure with simultaneous slight rubbing.

This is addressed by the document DE 26 19 410. In it there is a description of a sole as a foot support for massage therapy, which is set up for bearing a massage element. Provided for this over part of the entire surface area on the upper side of the sole are depressions, in which massage knobs can be alternately inserted and kept by the depressions. The depressions may be arranged uniformly or on the basis of patterns corresponding to associated reflex zone points of the sole of the foot. The massage elements may be of different shapes and sizes.

However, with this arrangement it has proven to be disadvantageous that the multiplicity of possible positioning locations for the massage elements that forms the basis for the versatility of the system requires a precise knowledge of the position of the foot reflex zones to be acted on, in order to provide the massage elements at a suitable place. A final customer cannot be expected to have this knowledge. Furthermore, the unoccupied depressions become soiled, which—apart from hygiene concerns—can cause problems when inserting the massage elements. The insertion of the longer massage elements described in the document, which extend over a number of depressions, is restricted by the shape of the foot bed. Here it is either necessary to use massage elements specifically adapted for the respective position and the shape there of the foot bed or there is the risk of the massage elements partly lifting off from the sole.

A further disadvantage of the arrangement is that the height of the massage elements cannot be adjusted continuously, but only in the graduations offered, by corresponding exchange of the massage elements. The pressure on the respective foot reflex zone can therefore only be adjusted in stages, for which purpose a relatively large number of different massage elements have to be kept in stock.

In WO 86/01381 there is a description of an insole for footwear with which a multiplicity of profile elements are connected. In this case, the height of at least some of the profile elements can be adjusted to the extent that they adapt themselves to the curvature of the foot. The adjustable profile elements comprise a pin connected to the insole, with a thread onto which a profile head with a covering cap can be screwed. The disadvantage of this arrangement is that the foot stands completely on adjacent profile elements, which leads to low wearing comfort, since the foot stands only on comparatively few points. This disadvantage could be overcome by a very large number of small profile elements, which however would lead to a considerable effort for the individual adjustment of the tread. Even with a comparatively small number of profile elements, an individual tread that is adapted to the shape of the foot is only possible with great effort in the adjustment of the profile elements.

In the document it is further proposed to provide the profile elements underneath the insole or on its underside. The foot is therefore offered a more uniform supporting surface by the insole, which increases the wearing comfort. The effort for adjusting the profile elements to the shape of the foot remains considerable, however, especially since now the insole has to be removed for the adjustment of the elements. Furthermore, in order to ensure a uniform supporting surface for the foot, the sole must be produced from a rather rigid material. This, however, prevents an increased pressure from being specifically built up in certain regions of the foot by protruding profile elements for local acupressure, since the rigid insole does not allow a sharply delimited pressure point.

In the document U.S. Pat. No. 5,682,690 there is proposed a shoe which includes a shoe sole comprising an upper sole, a lower sole and a midsole lying in between. In this case, the upper sole has holes, under which cavities are provided in the midsole. Massage pins protrude through the holes in the upper sole, reach with a lower end into the cavities of the midsole and are supported there in each case by a spring and a holding element lying thereover. The massage pins are therefore pressed by a placed-on foot counter to the spring force into the holes in the upper sole and into the cavities of the midsole. In this case, the prestressing of the springs can be adjusted individually by adjusting mechanisms which are accessible through corresponding holes in the lower sole. Furthermore, the document describes that individual massage pins can be removed as and when required.

By this means, the device makes it possible to adjust the pressure on individual regions of the sole of the foot individually. With this arrangement, however, the height of the massage pins by which they protrude beyond the upper sole can only be adjusted by exchanging the massage pins. In this case, the maximum length, and consequently the projection of the pins beyond the upper sole, tends to be small, since the receptacle for the pin, the spring and the tool receptacle are also provided as an extension of the pins, and the size of the overall construction is limited by the thickness of the midsole.

A further disadvantage of the massage unit is the rigid holding element for the spring, the adjusting element and the receptacle for the massage pin, arranged in a chamber in the midsole. The holding element, in which a thread for the adjustment of the spring tension is contained, is produced from a rather rigid plastics material and extends with its comparatively narrow cylindrical walls from the lower sole to the upper sole. In the region around the massage pin, the foot consequently no longer stands over the upper sole on a cushioning, elastic midsole but on the rigid, laterally sharply delimited holding element. In particular in the case of great local loading of the foot, for example in the region of the heel, this can lead to very high, and at least unpleasant, compressive loads, which significantly restricts the wearing comfort. In this case, the low wearing comfort is further exacerbated by the massage pins forming a small, sharply delimited supporting surface for the foot by their small diameter, and consequently leading to a highly punctiform pressure point on the sole of the foot, to which the relatively rigid material of the massage pins contributes.

If no massage is to take place, for instance at least in one region of the foot, the massage pin must be laboriously removed and kept for later use. In this case, a hole remains in the insole, through which dirt can penetrate into the chamber of the massage unit and to the region of the spring, which, apart from hygiene concerns, can lead to impairment of the spring and adjustment mechanism.

### GENERAL DESCRIPTION OF THE INVENTION

The invention is consequently based on the object of providing a shoe sole or footwear with a pressure massage function for the sole of the foot which makes simple adjustment of the massage pressure possible along with good wearing comfort.

A further object of the invention is to provide a shoe sole or footwear which makes possible a specific acupressure of certain desired acupressure points that can be easily adapted to the individual and/or temporary needs of the wearer.

This object is already achieved in an most surprisingly simple way by a shoe sole with a pressure massage function as claimed in claim 1, and by a shoe and a sandal as claimed in claims 27 and 28. Advantageous developments are the subject of the respective subclaims.

Accordingly, the invention provides a shoe sole with a pressure massage function, comprising a lower outsole, an upper insole or shoe insole and at least one adjustable pressure massage element between the outsole and the insole, the pressure massage element being accessible from below through the outsole through an aperture in the outsole, in order to carry out the adjustment of the pressure massage element, the pressure massage element having a stud extending transversely in relation to the shoe sole for bringing about the pressure massage on the foot. In this case, the stud is adjustable in height, at least between an upper active position, in which the stud produces an elevation in the insole and the pressure massage is thereby exerted on the foot, and a lower inactive position.

A shoe sole with the described pressure massage element makes it possible by the choice of an inactive or active position of the stud by the wearer for him to determine himself whether the pressure massage is to take place or not in a certain region of the foot. In this case, the height adjustment of the stud between the active position and the inactive position takes place with little effort from below or, in relation to the shoe from the outside, through the outsole, without elements of the shoe sole or of the pressure massage element having to be removed or exchanged and without exposing chambers in which dirt can be deposited. It is even possible to carry out the adjustment on the foot.

It has proven to be particularly advantageous for the achievable pressure distribution if the insole has in the region of the stud an aperture through which the stud protrudes beyond the insole in at least an upper active position. Consequently, the stud does not raise the insole with it during the adjustment into an active position, which at least in the case of the usually not permanently elastic insoles would lead to distortions and permanent deformations. Furthermore, the pressure point of the pressure massage element can therefore be spatially confined better than is possible by the comparatively rigid insole.

The pressure distribution and the function of the pressure massage element can be further optimized if the upper end of the stud is covered by a covering. In this case, the material and the shape of the covering can be adapted to the requirements imposed on the covering without any major restrictions, whereas in the case of a covering of the stud by the insole, these possibilities are limited on account of the additional requirements imposed on the insole.

In this respect, in a preferred embodiment of the invention the covering that is not integral with the insole is of a sheet-like formation, the covering being fixed between the insole and the outsole and resting on at least one stud of a pressure massage element. The stud and the entire pressure massage element are therefore covered over by the covering, which prevents the penetration of dirt. In this case, each pressure massage element may be assigned a separate covering, or a larger covering may be provided for a number of or all of the pressure massage elements.

In order to avoid a covering fixed alongside the stud from tearing directly alongside the stud during a height adjustment of the stud, it may be advantageously provided that a non-fixed region is provided between the fixing region of the covering and the region of the covering resting on the at least one stud. Here, the covering is consequently freely movable and can follow the movement of the stud.

An adequately large adjusting range of the stud can be achieved by the covering being permanently elastic, at least in the non-fixed region.

The permanently elastic covering is preferably produced from a polymer material, in particular from an elastic or visco-elastic plastic, in particular polymer. A visco-elastic material advantageously combines the properties of a viscose liquid and an elastic solid. The material of the covering preferably has an extensibility to break of greater than 100%, in particular greater than 200% or 300% or even greater than 500%. Sorbothane® has proven to be a particularly suitable visco-elastic polymer. With respect to further material properties of Sorbothane®, reference is made to the web site www.sorbothane.com, the content of which is hereby made the subject of this disclosure by reference.

A suitable material for the permanently elastic region, but also for an altogether permanently elastic covering, is therefore for example Sorbothane®. However, polyurethane or Neopren® may also be used. In this case, the production of an altogether permanently elastic covering offers the advantage of being producible in one process from a particularly uniform, one-layer material, and consequently at low cost.

If, on the other hand, the covering is produced as more than one layer from a combination of permanently elastic materials, different material properties, such as for example particularly good elasticity and high tear strength or good damping effect and durability, for example of the externally accessible layer, can be combined and consequently optimized for the covering.

It has proven to be particularly advantageous for wearing comfort if, in a lower inactive position of the stud, the covering in the non-fixed region lies with surface-area contact between the outsole and the insole and if, in an upper active position of the stud, the covering is stretched between the region resting on the stud and the fixing region, in which the covering is fixed, a continuous and elastic transition being formed by the covering between the region resting on the stud and the fixing region. In the active position, the foot consequently does not stand on a spatially narrowly confined stud with sharp lateral delimitations but on an elevation formed by the stud and the covering with sloping-down flanks, with smooth and gentle transitions between the flanks and the supporting region on the stud or with respect to the insole. In this case, the flanks are formed by the elastic extending region of the covering, in particular without a rigid material being used as a backing in this region. The degree of steepness of the flanks can be optimized by the geometry of the non-fixed region for best-possible wearing comfort.

In the upper active position, the covering also exerts a prestress on the stud, so that it is preferably possible to dispense with further, fault-susceptible, for example metallic, spring elements. The stud is advantageously secured by the prestress and the associated friction with the covering against unintentionally becoming unscrewed of its own accord.

High wearing comfort, in particular in the inactive position of the stud, can be achieved by the stud with the covering terminating in its lower inactive position under the aperture of the insole or substantially flush with the aperture of the insole. The insole and the covering therefore form a largely continuous surface area without disturbing transitions. The support for the foot then largely corresponds to the support on a conventional shoe sole not provided with pressure massage elements.

An additionally cushioning effect can be achieved by means of the stud, both in the active position and in the inactive position, by the covering over the stud having a thickened supporting region and/or a permanently elastic material which has a cushioning and/or restoring spring action being arranged between the covering and the stud.

The securement of the stud advantageously takes place by a plate-shaped holding element, which is integrated between the insole and the outsole, the holding element having an aperture for receiving the stud. The plate-shaped construction thereby makes a small thickness of the holding element possible, which makes integration possible even in comparatively thin shoe soles.

The reception of the stud in the holding element is designed in such a way that it makes a height adjustment of the stud possible. According to the invention, this can be achieved by the aperture of the holding element having a threaded guide or a sliding/snap-in guide and the stud having a corresponding threaded region or a corresponding snap-in region. In this case, the threaded guide in particular makes a continuous adjustment of the stud possible between the inactive position and the maximum active position. In the case of the sliding/snap-in guide, intermediate positions are possible in addition to the two end positions, so that the height of the stud can be changed in stages.

The holding element is arranged between the insole and the outsole. It is preferred for the holding element to be held on its lower area by the outsole overlapping the holding element. The outsole offers the holding element a durable supporting surface under the compressive and tensile stresses caused by the walking movement in the shoe sole, in the pressure massage elements and at the transitions from the shoe sole to the pressure massage element.

The adjustment of the stud takes place externally from the outsole. For this purpose, a corresponding adjusting tool may be used. As a counterpart to this tool, a complementary tool receptacle may be arranged at the lower end of the stud for receiving the adjusting tool for adjusting the stud.

In a refinement of the invention, the outsole has as an extension of the stud an aperture through which the tool receptacle of the stud is accessible. The aperture in this case preferably has the same diameter or a slightly larger diameter than the tool receptacle and the stud. The pressure massage element can therefore be adjusted from outside without great effort through the aperture. In the event of a defect of the stud or of the adjusting mechanism, the stud and the tool receptacle can be removed through the aperture and, if need be, replaced.

Sharp corners at the transition from the holding element to the shoe sole can be avoided by the holding element having a circular or oval outer contour. This effectively avoids the holding element becoming detached by the permanent tensile and compressive loading in the region of sharp-edged corners, in which the tensile and compressive stresses are particularly high.

For the production of a shoe sole with an integrated pressure massage element, it has proven to be advantageous if the pressure massage element with the holding element, the stud, the tool receptacle and the covering are provided as a unit and can be integrated as such in the shoe sole in the production process. For this purpose, in a preferred embodiment of the invention, the outer contour of the covering corresponds to the outer contour of the holding element, the covering being adhesively bonded in certain regions to the upper area of the holding element, or vulcanized onto it in certain regions.

The integration of the pressure massage element in the shoe sole may take place by a flange-shaped region being formed on the holding element, at least in certain regions, peripherally at the outer contour, and by the holding element being thereby firmly connected to the shoe sole. The flange-shaped region offers a large contact surface between the holding element and the shoe sole, and consequently the possibility of a connection that can withstand very high loading.

According to one embodiment of the shoe sole, the aperture in the insole is configured in such a way that the covering above the stud and the non-fixed region is not covered by the insole and that the insole covers the covering in the fixing region of the covering and the edge of the holding element. Undesired pressure points on the foot in the region of the transition from the elastic shoe sole material to the rather rigid holding element or to the fixing region, possibly embrittled slightly by adhesive bonding, can therefore be avoided by the resting insole. The adjustability of the stud and the extension of the covering in an active position of the stud are not impaired thereby, by virtue of the aperture and the insole.

A specific, individual massage of the foot can be achieved by the shoe sole containing one or more, preferably three, pressure massage elements. In this case, relevant regions of the foot, for example addressed foot reflex zones, can be acted on when the pressure massage elements are arranged in the regions of the foot that are advantageous for acupressure and/or foot reflex zone massage, the position of the foot over an ergonomically formed foot bed of the shoe sole being fixed in relation to the positions of the pressure massage elements. The arrangement makes it possible to provide a universal shoe sole which, by means of a relatively large number of pressure massage elements, can act on at least a majority of the foot reflex zones. It also presents the possibility of offering special shoe soles in which a small number of pressure massage elements are provided for the specific massage of a small number of foot reflex zones and the suitable shoe sole can be selected to correspond to the desired massage. For instance, it is possible to offer shoe soles with a pressure massage function which act for example only in the medial portion of the longitudinal arch of the foot and so, in accordance with foot reflex massage, primarily influence the spinal column. This offers a massage system, with which operating errors are to the greatest extent ruled out, to a final customer without specific knowledge of the foot reflex zones.

The universal suitability of the shoe sole corresponding to shoe soles without a pressure massage function can be achieved by the outsole being produced from a flexible and shock-absorbent material. Apart from the aperture, there are no significant additional requirements imposed on the outsole, so that tried-and-tested materials and constructions can be used.

In a preferred refinement of the invention, a midsole of a cork/rubber mixture or of a synthetic material, preferably of polyurethane, rubber or EVA, which has one or more receiving regions for receiving the pressure massage elements, is arranged between the outsole and the insole. The pressure massage elements can in this way be integrated in the receptacles of the midsole. By its cushioning effect, the midsole thereby increases the wearing comfort. Furthermore, the foot bed, which fixes the position of the foot in relation to the pressure massage elements, may be formed into the midsole.

If the outsole is produced from a layer of velour or from a layer of microfibers, a durable and hygienic contact layer with respect to the foot is obtained. The materials thereby effectively cushion the foot from the regions arranged thereunder of the pressure massage elements.

A durable construction of the pressure massage elements is achieved by the holding element and/or the stud being produced from plastic or from metal.

The invention also provides a shoe, comprising a shoe sole with a pressure massage function. By the integration of the shoe sole with the pressure massage function in a shoe, the previously described advantages of the invention become usable for a final customer. The versatility of such shoes with respect to area of use, shape and color can thereby correspond to the versatility of today's shoes without a pressure massage function.

The invention also provides a sandal, comprising a shoe sole with a pressure massage function. Also in this way, the previously described advantages of the invention become usable for a final customer. In this case, the open form of the sandal does not lead to any impairment of the function of the pressure massage elements, in particular since the elements are sealed by the covering, and are consequently protected to the greatest extent from penetrating moisture and dirt.

The invention is explained more precisely below on the basis of the accompanying figures. The same designations thereby refer to identical or similar parts.

### BRIEF DESCRIPTION OF THE FIGURES

In the figures:

FIG. 1 shows a schematic section through a shoe sole according to the invention with pressure massage elements

FIG. 2 shows a schematic section through a pressure massage element in the active position

FIG. 3 shows a schematic section through a pressure massage element in the inactive position

FIG. 4 shows a holding element in plan view

FIG. 5 shows a schematic representation of a sliding/snap-in guide

FIG. 6 shows a schematic longitudinal section through a shoe with an embodiment according to the invention of the shoe sole with pressure massage elements

FIG. 7 shows a schematic longitudinal section through a sandal with an embodiment according to the invention of the shoe sole with pressure massage elements

### DETAILED DESCRIPTION OF THE INVENTION

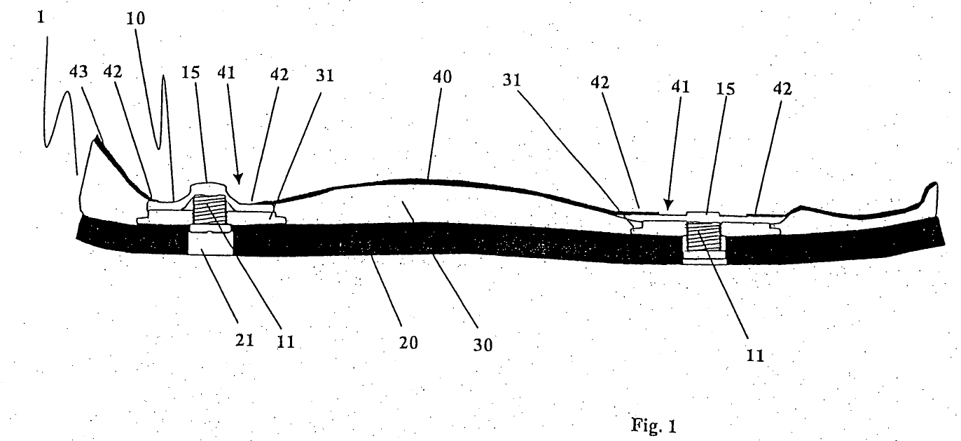

FIG. 1 shows a section through a shoe sole 1, which has an outsole 20 with apertures 21, a midsole 30 and an insole 40. The insole 40 is adapted in its shape to the midsole 30, which at the same time forms the foot bed 43. Provided in the midsole 30 is a recess, in which a pressure massage element 10 can be integrated. The pressure massage element 10 is connected via the receiving region 31 of the midsole 30 to the latter in the customary way. The pressure massage element 10 is covered over from below by the outsole 20 and is held by the latter. Apertures 41 are provided in the insole 40. These apertures 41 are closed from the side on which the pressure massage element 10 is located by a permanently elastic covering 15 of Sorbothane®. In the region of overlap 42 of the insole 40, the covering 15 of the pressure massage element 10 is covered over by the material of the insole 40. This provides additional cushioning of the foot in this region, both in the active position and in the inactive position.

The pressure massage element 10 shown here can be activated or deactivated via the apertures 21 of the outsole 20 by means of a special tool via the tool receptacle 13 of the stud 11. It goes without saying that the pressure massage element 10 may also be set to a likewise active intermediate position (not represented) with reduced acupressure effect, if so desired by the wearer.

As shown in the left part of FIG. 1, the activated pressure massage element 10 protrudes through the aperture 41 of the insole 40 in the direction of the interior of the shoe, owing to the screwed-in stud 11. As a result, the loading occurring during walking causes a pressure to be exerted on the rolling foot, massaging the foot at the acupressure points. If the pressure massage element 10 is deactivated, as shown in the right part of FIG. 1, the upper end of the stud 11 and the covering 15 lie flat with respect to the insole 40.

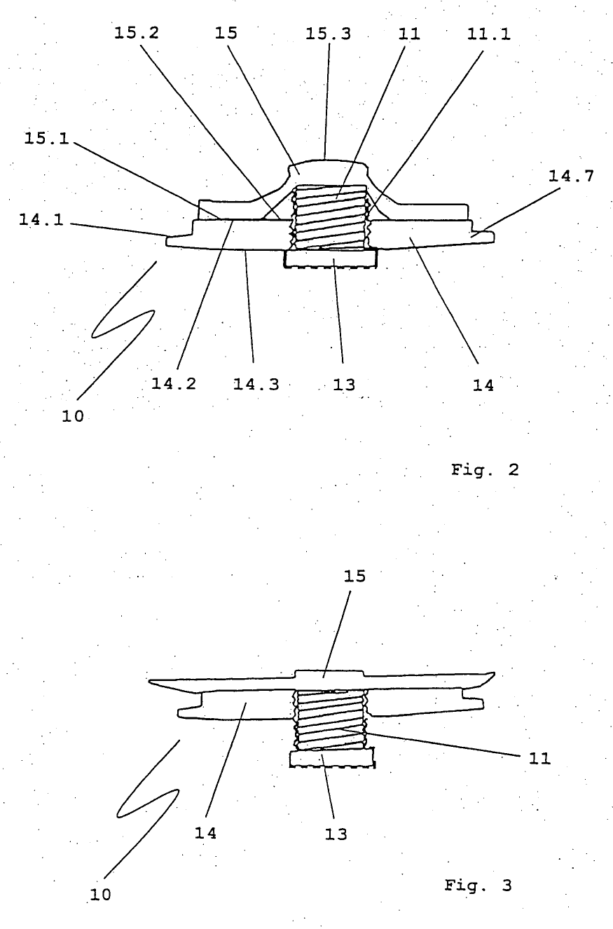

FIG. 2 shows a pressure massage element 10 in the active position. The pressure massage element 10 comprises a holding element 14, which has an aperture 14.5, which is provided with a threaded guide 14.4. The holding element 14 is connected via its edge 14.1 at the flange-shaped region 14.7 to the midsole 30 of the shoe sole 1. Indicated on the holding element 14 is an upper area 14.2, which corresponds to the fixing region 15.1 of the permanently elastic covering 15. The lower area 14.3 serves for securing the pressure massage element 10 and is covered over in the bond with the outsole 20. Provided directly at the aperture 14.5 of the holding element 14, running around peripherally, is a non-fixed region 15.2, in which the permanently elastic material of the covering 15 is not adhesively bonded with the holding element 14. This allows the covering 15 to lift off from the holding element 14 in the active position of the stud 11.

The activation and deactivation of the pressure massage element 10 takes place by the turning of a special tool in the tool receptacle 13 of the stud 11.

As a result, the stud 11 is guided upward or downward in its position, whereby the intensity of the massaging action can be adjusted. The stud 11 consists of metal or plastic and is flattened at its upper end. The permanently elastic covering 15 rests on this flattened part of the stud 11. The covering 15 laterally terminates with the outer contour of the holding element 14.

The permanently elastic covering 15 is thickened in terms of the thickness of the material in the supporting region 15.3. However, it is evident that the covering 15 may alternatively be formed as a planar layer, for example produced by simple punching out from a homogeneous sheet of permanently elastic material.

Similarly, it is conceivable to incorporate a further permanently elastic underlay between the stud 11 and the permanently elastic covering 15. In the active position of the pressure massage element 10, the permanently elastic covering 15 is stretched between the supporting region 15.3 and the region in which the covering 15 is connected to the holding element 14. As a result, the covering 15 creates a continuous and elastic transition between the supporting region 15.3 and the fixing region 15.1.

FIG. 3 shows a pressure massage element 10 in the inactive position. In this case, the stud 11 can be seen in its lower position. The permanently elastic covering 15 rests flat on the holding element 14 in the non-fixed region 15.2, as a result of which there is no elevation in the shoe sole 1.

The insole 40 (not represented here) thereby comes to lie with its overlapping region 42 over the covering 15 in such a way that it is congruent in terms of height with the supporting region 15.3. Consequently, the pressure massage element 10 can no longer be felt by the foot and the massaging action is therefore suspended.

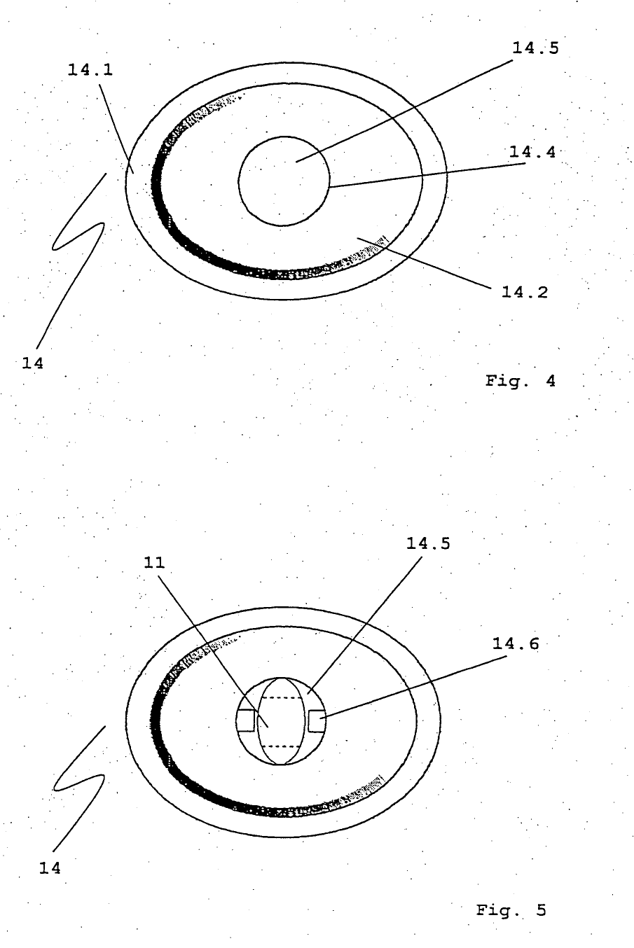

FIG. 4 shows a holding element 14 in plan view. The holding element 14 comprises a flat disk, which has a peripheral edge 14.1. The holding element 14 is connected via this edge 14.1 to the midsole 30. Also represented is an upper area 14.2, with which the holding element 14 is adhesively bonded with or vulcanized on the covering 15.

The holding element 14 has in its center an aperture 14.5 for receiving the stud 11. The aperture 14.5 is represented here with a threaded guide 14.4. This allows the stud 11 to be adjusted in its height by means of a special tool via the tool receptacle 13.

FIG. 5 shows a schematic representation of a sliding/snap-in guide 14.6. In this case, snap-in lugs are arranged in the aperture 15 of the holding element 14 and have corresponding gaps in the stud 11. The stud 11 is ovaly formed in terms of its shape, in order that sufficient space is obtained for the corresponding gaps. With the special tool, the stud 11 is turned to a position in which the snap-in lugs and the corresponding gaps are not congruent and the height of the stud 11 is adjusted by pushing with the special tool. When the next gap in the stud 11 is reached, this position is fixed by renewed turning of the stud 11. In comparison with the threaded guide 14.4 of FIG. 4, here the height adjustment of the stud 11 is not possible infinitely variably, but takes place in predetermined stages.

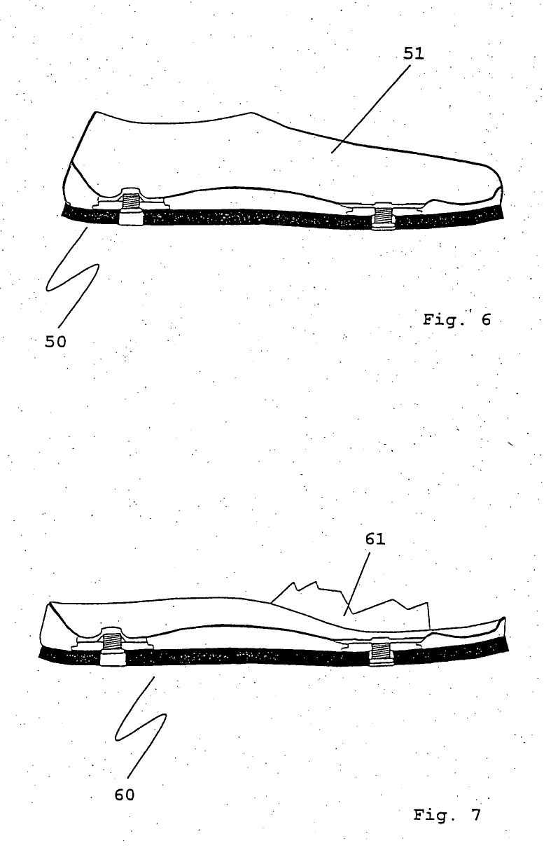

FIG. 6 shows a longitudinal section through a shoe with an embodiment according to the invention of the pressure massage element 10 and attached upper material 51.

FIG. 7 shows a longitudinal section through a sandal 60 with an embodiment according to the invention of the pressure massage element 10 and an attached strap 61.

Both refinements show that it is possible to incorporate the invention in commercially available footwear and thereby use the versatility of today's shoes without a pressure massage function—such as the area of use, shape and color. Even in the case of the sandal 60, the open form does not lead to an impairment of the function of the pressure massage elements 10, in particular since the pressure massage elements 10 are sealed by the covering 15, and are consequently protected to the greatest extent from penetrating moisture and dirt.

1. A shoe sole (1) with a pressure massage function, comprising
a lower outsole (20),
an upper insole (40)
and
at least one adjustable pressure massage element (10) between the outsole (20) and the insole (40),
the pressure massage element (10) being accessible from below through the outsole (20) through an aperture (21) in the outsole (20), in order to carry out the adjustment of the pressure massage element (10), the pressure massage element (10) having a stud (11) extending transversely in relation to the shoe sole (1),
characterized in that
the pressure massage on the foot,
the stud (11) being adjustable in height, at least between an upper active position, in which the stud (11) produces an elevation in the insole (40) and the pressure massage is thereby exerted on the foot, and a lower inactive position, in which the massage effect is suspended.

2. The shoe sole (1) as claimed in claim 1, wherein the insole (40) has in the region of the stud (11) an aperture (41) through which the stud (11) protrudes beyond the insole (40) in at least an upper active position.

3. The shoe sole (1) as claimed in one of the preceding claims, wherein the upper end of the stud (11) is covered by a covering (15).

4. The shoe sole (1) as claimed in one of the preceding claims, wherein the covering (15) is of a sheet-like formation, the covering (15) being fixed between the insole (40) and the outsole (20) and resting on at least one stud (11) of a pressure massage element (10).

5. The shoe sole (1) as claimed in one of the preceding claims, wherein a non-fixed region (15.2) is provided between the fixing region (15.1) of the covering (15) and the region (15.3) of the covering (15) resting on the at least one stud (11).

6. The shoe sole (1) as claimed in one of the preceding claims, wherein the covering (15) is permanently elastic, at least in the non-fixed region (15.2).

7. The shoe sole (1) as claimed in one of the preceding claims, wherein the covering (15) is produced from a permanently elastic material, preferably from Sorbothane®, from polyurethane or from Neopren®.

8. The shoe sole (1) as claimed in one of the preceding claims, wherein the covering (15) is produced as more than one layer from a combination of permanently elastic materials.

9. The shoe sole (1) as claimed in one of the preceding claims, wherein, in a lower inactive position of the stud (11), the covering (15) in the non-fixed region (15.2) lies with surface-area contact between the outsole (20) and the insole (40) and wherein, in an upper active position of the stud (11), the covering (15) is stretched between the region (15.3) resting on the stud (11) and the fixing region (15.1), in which the covering (15) is fixed, a continuous and elastic transition being formed by the covering (15) between the region (15.3) resting on the stud (11) and the fixing region (15.1).

10. The shoe sole (1) as claimed in one of the preceding claims, wherein the stud (11) with the covering (15) terminates in its lower inactive position under the aperture (41) of the insole (40) or substantially flush with the aperture (41) of the insole (40).

11. The shoe sole (1) as claimed in one of the preceding claims, wherein the covering (15) over the stud (11) has a thickened supporting region (15.3) and/or a permanently elastic material which has a cushioning and/or restoring spring action is arranged between the covering (15) and the stud (11).

12. The shoe sole (1) as claimed in one of the preceding claims, wherein the pressure massage element (10) has a plate-shaped holding element (14), which is integrated between the insole (40) and the outsole (20), the holding element (14) having an aperture (14.5) for receiving the stud (11).

13. The shoe sole (1) as claimed in one of the preceding claims, wherein the aperture (14.5) of the holding element (14) has a threaded guide (14.4) or a sliding/snap-in guide (14.6) and the stud (11) has a corresponding threaded region (11.1) or a corresponding snap-in region (11.3).

14. The shoe sole (1) as claimed in one of the preceding claims, wherein the holding element (14) is held on its lower area (14.3) by the outsole (20) overlapping the holding element (14).

15. The shoe sole (1) as claimed in one of the preceding claims, wherein a tool receptacle (13) is arranged at the lower end of the stud (11) for receiving an adjusting tool for adjusting the stud (11).

16. The shoe sole (1) as claimed in one of the preceding claims, wherein the outsole (20) has as an extension of the stud (11) an aperture (21) through which the tool receptacle (13) of the stud (11) is accessible, which aperture preferably has the same diameter or a slightly larger diameter than the tool receptacle (13) and the stud (11).

17. The shoe sole (1) as claimed in one of the preceding claims, wherein the holding element (14) has a circular or oval outer contour.

18. The shoe sole (1) as claimed in one of the preceding claims, wherein the outer contour of the covering (15) corresponds to the outer contour of the holding element (14) and the covering (15) is adhesively bonded in certain regions to the upper area (14.2) of the holding element (14), or is vulcanized onto it in certain regions.

19. The shoe sole (1) as claimed in one of the preceding claims, wherein a flange-shaped region (14.7) is formed on the holding element (14), at least in certain regions, peripherally at the outer contour, and wherein the holding element (14) is thereby firmly connected to the shoe sole (1).

20. The shoe sole (1) as claimed in one of the preceding claims, wherein the aperture (41) in the insole (40) is configured in such a way that the covering (15) above the stud (11) and the non-fixed region (15.2) is not covered by the insole (40) and wherein the insole (40) covers the covering (15) in the fixing region (15.1) of the covering (15) and the edge (14.1) of the holding element (14).

21. The shoe sole (1) as claimed in one of the preceding claims, wherein the shoe sole (1) contains one or more, preferably three, pressure massage elements (10).

22. The shoe sole (1) as claimed in one of the preceding claims, wherein the pressure massage elements (10) are arranged in the regions of a foot that are advantageous for acupressure and/or foot reflex zone massage, the position of the foot over an ergonomically formed foot bed (43) of the shoe sole (1) being fixed in relation to the positions of the pressure massage elements (10).

23. The shoe sole (1) as claimed in one of the preceding claims, wherein the outsole (20) is produced from a flexible and shock-absorbent material.

24. The shoe sole (1) as claimed in one of the preceding claims, wherein a midsole (30) of a cork/rubber mixture or a synthetic material, preferably of polyurethane, rubber or EVA, which has one or more receiving regions (31) for receiving the pressure massage elements (10), is arranged between the outsole (20) and the insole (40).

25. The shoe sole (1) as claimed in one of the preceding claims, wherein the insole (40) is produced from a layer of velour or from a layer of microfibers.

26. The shoe sole (1) as claimed in one of the preceding claims, wherein the holding element (14) and/or the stud (11) is produced from plastic and/or from metal, preferably from lightweight metal.

27. A shoe (50), comprising the shoe sole (1) with a pressure massage function as claimed in one of claims 1 to 26.

28. A sandal (60), comprising the shoe sole (1) with a pressure massage function as claimed in one of claims 1 to 26.
